(19) United States
(12) Patent Application Publication
(10) Pub. No.: US 2006/0173223 A1
(43) Pub. Date: Aug. 3, 2006

(54) Method of increasing the carbon chain length of olefinic compounds

(76) Inventor: Jochem Van De Weerd, Pianello del Lario (IT)

Correspondence Address: SHELL OIL COMPANY, P O BOX 2463, HOUSTON, TX, 772522463, US

(21) Appl. No.: 10/808,001

(22) Filed: Mar. 24, 2004

(51) Int. Cl. C07C 11/00; C07C 1/00

(52) U.S. Cl. 585/16; 585/327

(57) ABSTRACT

According to the present invention there is provided a process of increasing the carbon chain length of an olefinic compound comprising the steps of providing a starting olefinic compound and subjecting it to hydroformylation to produce an aldehyde and/or alcohol with an increased carbon chain length compared to the starting olefinic compound. Optionally, the aldehyde that may form during the hydroformylation reaction is hydrogenated to convert it to an alcohol which has an increased carbon chain length compared to the starting olefinic compound. The alcohol with the increased carbon chain length is subjected to dehydration to produce an olefinic compound with an increased carbon chain length compared to the starting olefinic compound. The invention also relates to olefinic compounds produced by the process.

### FIELD OF THE INVENTION

This invention relates to a process of increasing the carbon chain length of olefinic compounds. The invention also relates to olefinic compounds produced by this process.

### BACKGROUND OF THE INVENTION

There is a high demand for longer chain α-olefins, especially even numbered α-olefins such as 1-hexene and 1-octene. 1-Hexene and 1-octene are used, amongst others, as co-monomers in polyethylene production where they serve as plasticizers, e.g., as co-monomers in the preparation of linear low-density polyethylene.

One method of producing olefins is through an olefin metathesis reaction. A disadvantage of this type of reaction is that it is difficult to control the reaction to produce only one specific olefin and the majority of olefins produced by this process are internal olefins. Metathesis reactions are accordingly not very suitable for preparing α-olefins such as 1-hexene or 1-octene. One type of metathesis reaction, namely ethenolysis between an internal olefin and ethylene, can potentially yield α-olefins, but the technology suffers from equilibrium and selectivity limitations. Furthermore, ethenolysis of an internal olefin would result in an olefin with a shorter carbon chain than the starting internal olefin.

1-Hexene can also be produced by means of trimerization of ethylene. Although this is a well-known method for producing 1-hexene, it has the disadvantage that C4, C8 and C10 impurities and polyethylene are also produced.

Fischer-Tropsch technology produces a large range of hydrocarbon products following an Anderson-Schulz-Flory distribution. This means that more 1-pentene is produced than 1-hexene. The market demand for 1-pentene is small with the result that most of the 1-pentene ends up in a fuel pool, resulting in a fuel alternative value. On the other hand 1-hexene is sold at a much higher value. The same is true for heptenes and butenes. It is believed that with the process of the present invention 1-butene, 1-pentene, and/or 1-heptene can undergo controlled chain growth reactions to yield 1-hexene and/or 1-octene.

### SUMMARY OF THE INVENTION

The present invention is a novel process of increasing the carbon chain length of olefinic compounds, including and especially α-olefins. Accordingly, shorter α-olefins such as 1-pentene can be converted to 1-hexene.

According to the present invention there is provided a process of increasing the carbon chain length of an olefinic compound comprising the steps of:
a) providing a starting olefinic compound and subjecting it to hydroformylation to produce an aldehyde and/or alcohol with an increased carbon chain length compared to the starting olefinic compound;
b) optionally, hydrogenating the aldehyde that forms during the hydroformylation reaction to convert it to an alcohol which has an increased carbon chain length compared to the starting olefinic compound; and
c) subjecting the alcohol with the increased carbon chain length to dehydration to produce an olefinic compound with an increased carbon chain length compared to the starting olefinic compound.

In this specification the term olefinic compound means an olefin and/or a substituted olefin which includes one or more heteroatoms which are neither carbon nor hydrogen. It will be appreciated that the chain length may be increased by, for example, lengthening the only carbon chain in the case of unbranched linear compounds, lengthening the longest carbon chain or a branch carbon chain in the case of a branched carbon chain product, or by the formation of a branch carbon chain or the formation of an additional branch carbon chain.

In one embodiment, the process is a process for producing linear unbranched olefins, preferably α-olefins, preferably α-olefins with an even number of carbon atoms, preferably 1-hexene and/or 1-octene. In another embodiment, the process is one wherein the carbon chain length of an olefinic compound, preferably an α-olefinic compound, with an odd number of carbon atoms is increased by one carbon to an olefinic, preferably α-olefinic, compound with an even number of carbon atoms.

Preferably the starting olefinic compound comprises an olefin, preferably an olefin with a single carbon-carbon double bond. Preferably the starting olefin is an unbranched linear olefin, preferably an α-olefin, and often it will be an α-olefin with an odd number of carbons in the carbon chain, such as 1-pentene and/or 1-heptene.

In one embodiment of the invention pentenes, preferably 1-pentene, may be converted to hexenes, preferably 1-hexene. Alternatively or additionally heptenes, preferably 1-heptene, may be converted to octenes, preferably 1-octene.

In one embodiment of the invention a Fischer-Tropsch derived feed stream containing one or more olefins, preferably α-olefins, may be used as a source of the starting olefinic compound. Preferably, the feed stream contains a significant concentration of olefins having an odd number of carbon atoms.

It will be appreciated that the process can be used to obtain controlled carbon chain growth of olefinic compounds and the process may be repeated to obtain chain growth of the formed olefinic compound. That is, for example, linear butenes, preferably 1-butene, as starting olefin may undergo chain growth by one carbon to be converted to 1-pentene which may then be converted to 1-hexene.

### DETAILED DESCRIPTION OF THE INVENTION

Hydroformylation of olefinic compounds to produce aldehydes and/or alcohols with an increased carbon chain length is well known and can be carried out in many different and known ways. This step and the different options available are accordingly not described in detail in this specification.

It will be appreciated that during hydroformylation of an olefin, hydrogen and carbonyl are added to the carbon atoms across a double bond to yield a compound with an increased carbon chain length compared to the starting olefin. When the carbon atom of the carbonyl group is bound to hydrogen, an aldehyde is formed. Some aldehydes may, depending on the type of catalyst used, automatically convert to the corresponding alcohol by means of an in situ hydrogenation reaction. It is believed that in the case of a catalyzed hydroformylation reaction, a leaving group (usually in the form of the catalyst or a derivative thereof) will be bound to the carbonyl group. If the leaving group is replaced with hydrogen, an aldehyde forms. Alternatively, if the leaving group is replaced with hydrogen and hydrogenation takes place, an alcohol forms.

In one embodiment of the invention the hydroformylation step may be carried out by reacting the olefinic compound with CO and H₂ in the presence of a suitable catalyst and under suitable conditions. The catalyst may comprise a suitable Rh catalyst [e.g. Rh(acetylacetonate)(CO)₂] in combination with triphenyl phosphine), but preferably it comprises a suitable cobalt catalyst, for example cobalt with the ligand eicosyl phoban. Other possible catalysts include palladium catalysts used in the production of alcohols by reaction of olefins with syngas under hydroformylation conditions such as described in U.S. Pat. Nos. 6,037,506, 5,488,174, and 6,156,936, which are herein incorporated by reference, and catalysts used in the oxo process for making alcohols which is well-known in the art.

The reaction may be carried out in a temperature range from about 25 to about 250° C., preferably from about 100 to about 200° C. The reaction may be carried out at a pressure from about 10 to about 100 barg, preferably about 60 to about 90 barg.

In a preferred embodiment of the invention the catalyst and reaction conditions are selected to obtain a high selectivity of n-alcohols as reaction product when an olefin, preferably an α-olefin, is used as the starting olefinic compound. Preferably, a selectivity of at least about 80% is obtained, possibly even at least about 90%.

In cases where significant amounts of aldehyde is produced during the hydroformylation, it is preferred to include a hydrogenation step to convert the aldehyde to an alcohol. Where no significant amount of aldehyde forms during the hydroformylation, a hydrogenation step may not be required. The hydrogenation may comprise reacting the aldehyde in a solvent or neat with H₂ in the presence of any suitable hydrogenation catalyst (for example, Pd—C, Pt—Al₂O₃, Cu/Cr, Ni—Al₂O₃, etc). This is a well known process and is accordingly not described in detail in this specification.

Removal of unwanted products may take place at any stage prior to or after the dehydration process step. Preferably, unwanted alcohols or aldehydes are removed prior to the dehydration step.

Where branched alcohols or aldehydes are produced during the hydroformylation step and optionally the hydrogenation step, and a linear α-olefinic compound is desirable, such branched alcohols or aldehydes may be removed, for example by distillation, before the dehydration step to improve the selectivity to linear olefinic compounds. Unwanted aldehydes can be removed, e.g. by distillation, prior to the hydrogenation step.

A suitable feedstock for hydroformylation may contain a single olefin or may be a mixture of olefin isomers. It will be appreciated that each olefin isomer contained in a mixed olefin feed may form different isomers of aldehydes or alcohols during hydroformylation, For example, 1-pentene may form 1-hexanol or 2-methyl-1-pentanol, depending upon to which carbon atom of the double bond the CO group bonds during hydroformylation. Similarly, the hydroformylation of 1-heptene may yield 1-octanol or 2-methyl-1-heptanol and other olefins may yield the corresponding alcohols. The same principle applies where the hydroformylation product is an aldehyde.

It is well known in the art of hydroformylation processes that although extreme efforts are undertaken to selectively produce a specific isomer as product, a significant concentration of the other isomer also forms. This is the case with all known hydroformylation catalyst types and would occur in hydroformylation reactions irrespective of which catalyst is used.

According to the present invention, the alcohol isomers formed by hydroformylation (and optionally hydrogenation) are dehydrated to yield their corresponding olefinic compound isomers. Where a pure product, for example comonomer grade α-olefin is the desired product, these olefinic compound isomers must be separated from each other. The olefinic compound isomer mixtures may be purified by distillation processes. However, certain of these olefinic compound isomers have boiling points so close to each other that distillation becomes extremely complex. For example, the boiling points of 1-hexene and 2-methyl-1-pentene are 63.5 and 62.1° C., respectively. To separate these close boiling compounds by distillation is extremely capital intensive as distillation columns with a large number of distillation stages are required. Thus, dehydration products like 1-hexene cannot be separated from 2-methyl-1-pentene and 2-ethyl-1-butene to produce a pure 1-hexene product in a fashion which is commercially feasible.

It has been found that the desired olefin can be produced in high purity by removing unwanted compounds prior to the dehydration step, preferably by distillation of the alcohol and/or aldehyde produced by hydroformylation (and optionally hydrogenation) before dehydration thereof, to produce a high purity olefinic compound (e.g. an α-olefin) with increased carbon chain length when compared to that of the starting olefinic compound. By distillation of the alcohol product, prior to dehydration thereof, close boiling point olefinic compound isomers, for example 2-methyl-1-pentene and 2-ethyl-1-butene are not produced, which enables production of high purity olefins (e.g. α-olefins).

Thus, according to this invention, desired olefinic compounds (especially α-olefins) can be produced from shorter chain olefinic compounds, in high purities, especially by distillation of the alcohol product prior to dehydration thereof. Thus, according to this invention, desired α-olefinic compounds (especially α-olefins) can be produced from shorter chain olefinic compounds in purities of greater than 95% of the desired isomer. More preferably, desired olefinic compounds (especially α-olefins) can be produced from shorter chain olefinic compounds (especially α-olefins) in purities of greater than 98% of the desired isomer.

Any suitable dehydration process may be used to convert the alcohol with the increased carbon chain length, to the olefinic compound. In cases where the alcohol is a n-alcohol (or significant concentrations thereof are present), the dehydration process is preferably controlled to produce α-olefinic compounds.

Many different dehydration processes are known and they are accordingly not discussed in any detail herein. Preferably the dehydration is carried out under low acidity conditions and a low acidity catalysts support such as Al₂O₃, SiO₂, TiO₂, or ZrO₂ may be employed to afford a dehydration reaction at temperatures from about 200 to about 450° C., typically from about 250 to about 350° C., and at pressures from about 0 to about 30 barg, typically from about 0 to about 5 barg. The catalyst may comprise a gamma-alumina catalyst or a promoted alumina catalyst, for example CaO.Al₂O₃, Ca₂O₃.Al₂O₃.

This invention also relates to a product produced by the process substantially as described hereinabove.

### EXAMPLES

Experimental scouting work was carried on the dehydration of 1-decanol, and NEODOL-1 and Linevol-911 alcohols. The dehydration was carried out in a continuous reactor in the gas phase (at a weight hour space velocity, the mass flow of feedstock per hour per unit volume of catalyst [WHSV] of 1240 kg·m-3.h-1) using an aluminium oxide dehydration catalyst from Engelhard (AL-0104-T).

Well known side reactions in this chemistry are ether formation, olefin isomerization and oligomerization. In order to find the optimum dehydration conditions the initial experiments were carried out with pure (99%) 1-decanol, the results of which are summarized in Table 1.

TABLE 1
Dehydration of 1-decanol to 1-decene (based on gas chromatographic
data in GLC-Area %, a relative measure of mass [wt. %])

| Temp. | | didecyl | total | selectivity |
|---|---|---|---|---|
| (° C.) | Conversion | ether | decenes | to 1-decene |
| 282 | 79.3% | 26.7% | 51.4% | 97.4% |
| 301 | 87.6% | 12.8% | 73.6% | 96.5% |
| 313 | 96.2% | 1.9% | 92.5% | 95.0% |
| 325 | 99.3% | 0.2% | 97.3% | 93.1% |

As can be seen in Table 1 the best results (high conversion, low ether formation) were obtained at a temperature of 325° C. At this temperature, >99% of the alcohol is converted, the yield of decenes is 97%, and the selectivity to 1-decene is 93% (based on total decenes). The two most abundant other decenes were tentatively identified as cis-2-decene (4.1%) and trans-2-decene (1.6%). The effective conversion (per pass) of 1-decanol to 1-decene is greater than 91%. It may be possible to leave the minor amounts of residual alcohol, ether, and isomeric decenes in the product.

The same conditions to the dehydration of the alcohols Neodol-1 and Linevol-911 alcohol which were produced by conversion by hydroformylation of either internal decenes or a mixture of internal octenes, nonenes, and decenes made by metathesis of a mixture of C₄-C₅₀ (on average) internal olefins prepared by isomerizing a mixture of the corresponding alpha-olefins. The dehydration of these alcohols proved to proceed just as smoothly as observed for 1-decanol at 325° C. The conversion was again about 99% and the ether formation was as low as 0.2%.

The dehydration of Neodol-1 alcohol gave a mixture of undecenes with an 1-undecene content of 77.0%. Given the fact that the starting material Neodol-1 alcohol contained 83.5% 1-undecanol, the selectivity is comparable (92-93%) to the experiment with 1-decanol.

The dehydration of Linevol-911 alcohol, with a normality (1-alcohol content) of about 82% and containing about 19% C₉, 45% C₁₀, and 36% C₁₁, gave a mixture of nonenes, decenes, and undecenes. The mixture contained about 1% of starting material, 0.5% of ethers, about 15% of 1-nonene, 34% of 1-decene and 27% of 1-undecene. A total of about 76% 1-alkenes (the remainder being predominantly vinylidene olefins) means again a selectivity of 92.5%, as the starting material contained only 82% of 1-alcohols.

In conclusion, it can be stated that the dehydration of higher alcohols (C9-C11) to higher olefins proceeds smoothly with the dehydration catalyst at 325° C., showing high conversion (>99%), a higher effective conversion (per pass) of 1-alcohols to 1-alkenes (>91%), and a high selectivity to α-olefins (>92%). Although at lower temperatures the conversion and ether formation become unfavourable, the selectivity to 1-decene, based on total decenes, is better (some 97%). The unconverted alcohol and the ether may be recycled to the dehydration reactor to extinction in order to improve the overall selectivity to 1-decene. A higher conversion at a lower temperature might be obtained with a modified alumina catalyst.

I claim:

1. A process of increasing the carbon chain length of an olefinic compound comprising the steps of:
a) providing a starting olefinic compound and subjecting it to hydroformylation to produce an aldehyde and/or alcohol with an increased carbon chain length compared to the starting olefinic compound;
b) optionally, hydrogenating the aldehyde that forms during the hydroformylation reaction to convert it to an alcohol which has an increased carbon chain length compared to the starting olefinic compound; and
c) subjecting the alcohol with the increased carbon chain length to dehydration to produce an olefinic compound with an increased carbonchain length compared to the starting olefinic compound.

2. The process of claim 1 wherein the carbon chain length of an olefinic compound with an odd number of carbon atoms is increased by one carbon to an α-olefinic compound with an even number of carbon atoms.

3. The process of claim 2 wherein 1-pentene is converted to 1-hexene.

4. The process of claim 2 wherein 1-heptene is converted to 1-octene.

5. The process of claim 1 wherein the starting olefinic compound comprises an unbranched linear α-olefin with a single carbon-carbon double bond.

6. The process of claim 1 wherein a Fischer-Tropsch derived feed stream containing one or more olefins is used as the starting olefinic compound.

7. The process of claim 1 wherein the hydroformylation is carried out by reacting the olefinic compound with carbon monoxide and hydrogen in the presence of a suitable catalyst.

8. The process of claim 1 wherein significant amounts of aldehyde are produced during hydroformylation and the process includes the step of hydrogenating the aldehyde to convert it to an alcohol which has an increased carbon chain length compared to the starting olefinic compound.

9. The process of claim 1 which includes the removal of unwanted products before or after the dehydration step.

10. The process of claim 9 where unwanted alcohols or aldehydes are removed prior to the dehydration step.

11. An olefinic compound produced by the process of claim 1.
